(19) United States
(12) Patent Application Publication
(10) Pub. No.: US 2025/0099949 A1
(43) Pub. Date: Mar. 27, 2025

(54) BRIDGED CATALYSTS AND SYNTHESIS AND USES THEREOF

(71) Applicant: PMC Organometallix, Inc., Mount Laurel, NJ (US)

(72) Inventors: Kevin John ROSS, Rockwood (CA); Wayne MEYER, Louisville, KY (US); Gene Kelly NORRIS, West Chester, OH (US)

(73) Assignee: PMC Organometallix, Inc., Mount Laurel, NJ (US)

(21) Appl. No.: 18/786,490

(22) Filed: Jul. 27, 2024

Related U.S. Application Data
(60) Provisional application No. 63/515,879, filed on Jul. 27, 2023.

(51) Int. Cl. B01J 31/22; B01J 37/06; B01J 37/08; C07B 41/12; C07B 43/00

(52) U.S. Cl. CPC B01J 31/22; B01J 37/06; B01J 37/08; C07B 41/12; C07B 43/00; B01J 2523/43

(57) ABSTRACT

The present invention relates to catalyst compositions. More particularly, the present invention relates catalyst compositions comprising at least one alkyl bridge group. The catalyst of the present invention has been shown to provide higher filterability than provided by non-bridged catalysts when used in ester reactions, and provide lower extractability than provided by non-bridged catalysts when used in urethane reactions. The present invention is expected to be catalytically active in the same way to all ester bonds, urethane bonds, silicones, siloxanes, organic tins.

### CROSS-REFERENCE TO RELATED APPLICATIONS

This application claims the benefit of U.S. Provisional Patent Application Ser. No. 63/515,879, filed on Jul. 27, 2023, which is incorporated herein by reference. Priority of U.S. Provisional Patent Application Ser. No. 63/515,879, filed on Jul. 27, 2023 is hereby claimed.

### BACKGROUND OF THE INVENTION

### 1. Field of the Invention

The present invention relates to catalyst compositions. More particularly, the present invention relates catalyst compositions comprising at least one bridging group. The catalyst of the present invention has been shown to provide higher filterability than provided by non-bridged catalysts when used in ester reactions and provide lower extractability than provided by non-bridged catalysts when used in urethane reactions. The present invention is expected to be catalytically active in the same way to all ester bonds, urethane bonds, silicones, siloxanes, organic tins.

### 2. General Background of the Invention

Binuclear compounds and methods of preparing them are known in the art. See CN 115716848. Bimetallic catalysts have been used to serve unique functions. In general, these functional bimetallic catalysts include mixed metals. The same metal bimetallic catalysts, especially symmetrical ones, don't generally serve a better reactive function than their monometallic counterparts. Additionally, monometallic catalysts are generally simpler and more cost-effective to make that bimetallic catalysts. However, there is a need for improved catalysts directed more toward retention or removal from the end-product rather than reactivity as a catalyst.

For many applications using tin-based catalysts one potential concern involves the loss of these tin-based materials from the finished article in downstream processing. Efforts have been made by use of multinuclear organotin catalyst but these materials tend to have lower activity and narrower effective range of use than their monomeric counterparts. See U.S. Pat. Nos. 5,436,357 and 5,561,205. Additionally, there is sometimes a need for the catalytic tin to be easily removed from the product.

The present invention can solve these issues. As shown, when an exemplary catalyst of the present invention was used for polyurethane, catalyst extraction was greatly reduced as compared to the extractability of its monomeric counterpart. Additionally, filterability was increased when using an exemplary bridged catalyst of the present invention s compared to the filterability of its monomeric counterpart.

### BRIEF SUMMARY OF THE INVENTION

The bridging catalyst of the present invention is preferably comprised of at least two monomeric components, having at least one bridging group between the two components, resulting in a binuclear compound. In the exemplary descriptions below, the catalyst butylstannoic acid (BSA) was used to create a catalyst compound having two BSA molecules with a simple bridging alkyl between the two tin centers. This exemplary novel catalyst is herein referred to as bis-BSA. The bridging catalyst, bis-BSA, was chosen as a simple representative of a class of catalysts that contain a simple bridging alkyl between two tin centers. Additionally, this form of bridged catalyst allows for simple comparison between the bridged catalyst and its monomer counterpart. However, it is expected that other catalysts or compounds will yield similar results as will other bridging groups. Additionally, while the present examples use bis-BSA having a simple bridging alkyl between two tin centers, other groups could also be added to the bridging group in addition to the two nuclear centers.

As shown in the examples below, bis-BSA provided similar catalytic activity to BSA in both esterification and polyurethane foam production. However, the bridging catalyst, bis-BSA, provides improved filterability when used as an esterification catalyst and also provides much lower extractability when used as a catalyst in polyurethane foam systems.

The alkyl-bridged materials described herein are based on tin-alkyl bonds. These bonds are not active in the catalytic process but are effective in increasing molecular weight of the catalytic system. This approach is not limited to monomeric species containing two tin sites with a single bridging group. It is expected that species with multiple tin sites, greater than two, based on single or multiple tin-terminated alkyl-based bridges should also behave in similar manner. Additionally, other compounds and alternate bridging groups are expected to yield similar results.

Exemplary catalysts of the present invention have been tested in esterification and polyurethane foam systems; however, the expectation is that the catalyst of the present invention will be catalytically active in the same way to ester bonds, urethane bonds, silicones, siloxanes, organic tins.

### DETAILED DESCRIPTION OF THE INVENTION

Monomeric catalysts are well known with acceptable catalytic performance. However, the bridged catalysts of the present invention have unexpected advantages of improvement in filterability and extractability. Additionally, the bridged catalysts of the present invention exhibit catalytic performance similar to a monomeric homologues.

The catalysts of the present invention provide a modest increase in molecular weight by use of a bridging alkyl, and provide similar reactivity to their monomeric counterpart, see Table 1. However, substantial differences in the ability to remove the catalyst result from ester-based reactions. See Tables 2 and 3.

The bis-BSA catalyst was also evaluated in another application: polyurethane foam. As outlined in Table 4, the bis-BSA and its non-bridged counterpart, BSA, both provide similar foaming activity to produce a polyurethane foam. The resulting foams were treated with dichloromethane (DCM) to explore the potential differences between the extractability of the two catalysts. As detailed in Table 5, the bis-BSA showed much lower extractability than did the monomeric material BSA.

Synthesis of HO(O)Sn(CH₂)₈Sn(O)OH (Hereinafter “Bis-BSA”):

A 250 mL flask was charged with NaOH (15 wt %, 72.3 g) and Tergitol 15-S-7 (0.02 g). The flask was fitted with mechanical stirring, a temperature probe, addition funnel, and condenser with a nitrogen purge. The addition funnel was charged with Cl₃Sn(CH₂)₈SnCl₃ (“bis-MBTC,” 28.68 g, 30.35% C1) and toluene (10 g). The bis-MBTC was added to the NaOH solution over 7 minutes while the temperature rose to 48° C. to give an off-white slurry. The temperature was heated to 66-69° C. and held for 3.5 hours. Deionized water (DI water) (100 mL) was added to the reaction slurry. The top water layer was removed by pipette and the slurry was washed with DI water (60 mL). The slurry was filtered on a Buchner funnel through Whatman #1. The reaction flask was rinsed with DI water (30 mL). This rinsate was used to wash the cake along with 2×10 mL DI water. The wetcake was dried and found to have 3.1% chloride. The powder was re-washed with DI water (70 mL×3 at 55-60° C., and 70 mL×2 at 30-40° C.). The slurry was filtered on a Buchner funnel. The wetcake was dried in a 60° C. oven 16 hours to give a tan powder (14.4 g, 52.71% Sn, 1.66% Cl).

### Example 1

An application test of bis-BSA as a catalyst in an esterification was conducted using adipic acid and 2-ethylhexanol.

A 250 mL flask was charged with adipic acid (78.92 g, 0.5400 mol), 2-ethylhexanol (EHA, 147.51 g, 1.133 mol), and bis-BSA (0.262 g). The flask was fitted with mechanical stirring, a temperature probe, and a moisture overhead collector with a 3-way valve to remove water from the system and return EHA to the reactor as needed. A steady flow of nitrogen (310 mL/min) was swept through the reactor. The flask was heated to 175° C., which was set at time zero. The temperature was then set to 210° C. and samples were taken every 30 minutes. The test was considered complete when the acid number was below 5 mg KOH/g. Tin loading based on the weight of the reactants was 0.061%. A similar experiment was conducted using BSA (Butylstannoic acid, or Fascat® 4100) at a tin loading of 0.060%.

TABLE 1
Extent of esterification reaction of adipic acid
and 2-ethylhexanol catalyzed by bis-BSA and
BSA catalysts as monitored by acid number.

| | bis-BSA | BSA |
|---|---|---|
| time | acid no. | acid no. |
| hours | mg KOH/g | mg KOH/g |
| 0.0 | 170 | 197 |
| 0.5 | 68.2 | 57.1 |
| 1.0 | 22.1 | 17.1 |
| 1.5 | 11.6 | 7.8 |
| 2.0 | 6.6 | 5.4 |
| 2.5 | 3.3 | 2.3 |

Tables 2 and 3 show the reduction in tin content via filtration from bis(ethylhexyl) adipate experiments.

TABLE 2
bis-BSA loaded at 0.18% (calc)

| Filtration Type | tin (ppm) | % reduction |
|---|---|---|
| none | 1477 | |
| 11 μm | 846 | 42.7 |
| 1.5 μm | 770 | 47.9 |

TABLE 3
BSA loaded at 0.18% (calc)

| filtration | tin (ppm) | % reduction |
|---|---|---|
| none | 1104 | |
| 11 μm | 935 | 15.3 |
| 1.5 μm | 872 | 21.0 |

The product, bis(ethylhexyl adipate), from both experiments was filtered by the methods described in Tables 2 and 3.

### Example 2

A polyurethane kit from Flinn Scientific, Inc. was used to test catalyst entrainment in a foam. The kit consisted of a Part A (polyol, amine, surfactant) and a part B (isocyanate blend). The procedure with the kit called for adding 20 mL of part B (ca. 27 g) to 20 mL of part A (ca 22 g) in an appropriate plastic, disposable container with enough capacity to hold the expanding foam.

A 1 L plastic beaker was charged with 20 mL part A. The catalyst (bis-BSA or BSA) was then added as a fine powder to the liquid in the beaker (both catalysts were added at the same tin loading). This mixture was then stirred vigorously with a wooden stirrer to provide a homogeneous slurry. Part B (20 mL) was then added while stirring. The stirring was continued until the reaction began (ca. 10-15 seconds) after which the wooden stirrer was removed. The foam rose to 3-4 cm above the top of the beaker. The foam was allowed to sit and cool overnight. A summary of these experiments is outlined in Table 4.

A 2 cm disc was cut from the center of the foam with a saw blade. This was cut into pieces with scissors into 0.5-1 cm pieces which were placed into a 50 mL gastight syringe with a cap at the bottom. Dichloromethane was added to fill the foam containing syringe. The cap was then quickly removed, a plunger installed and pushed to extract out the solvent into a glass bottle. Another portion of dichloromethane was added again to perform a second extraction in a method similar to the first extraction. The extracts were then diluted to the same final weight. The tin containing solvent was then extracted with 10.00 mL 6N HCl in a plastic bottle on a lab shaker for 20-30 minutes to allow migration of the tin-based species into the acid layer. The acid layer was then analyzed for tin by atomic absorption spectroscopy. These results are summarized in Table 5.

TABLE 4
Summary of Foam experiments

| | Part A g | | Catalyst | Catalyst | % Sn in | Foam | Foam sample |
|---|---|---|---|---|---|---|---|
| | polyol | Part B g | BSA | bis-BSA | formulation | (g) | extraction g |
| BSA | 22.15 | 27.7 | 0.509 | | 0.601 | 47.106 | 1.570 |
| Bis-BSA | 22.19 | 27.8 | | 0.537 | 0.590 | 47.923 | 1.546 |

TABLE 5
Summary of Foam extraction experiments

| | DCM 1st | DCM 2nd | extracted | diluted | 6N HCl | Extracted |
|---|---|---|---|---|---|---|
| | extraction g | extraction g | DCM g | to g | mL | Sn ppm |
| BSA | 43.00 | 16.31 | 43.74 | 70.58 | 10.00 | 522 |
| Bis-BSA | 43.87 | 16.33 | 45.39 | 70.08 | 10.00 | 233 |

As shown in the above examples, the catalysts of the present invention provide higher filterability than provided by non-bridged catalysts when used in ester reactions, and provide lower extractability than provided by non-bridged catalysts when used in urethane reactions.

What has been described and illustrated herein are examples along with some of their variations. The terms, descriptions and figures used herein are set forth by way of illustration only and are not meant as limitations. Many variations are possible within the spirit and scope of the subject matter, which is intended to be defined by the following claims—and their equivalents—in which all terms are meant in their broadest reasonable sense unless otherwise indicated.

1. A catalyst composition comprising:
at least two monomeric components; and
at least one bridging group between at least two nuclear centers of the at least two monomeric components.

2. A catalyst composition comprising:
at least two tin centers; and
at least one bridging group between the at least two tin-centers.

3. The catalyst composition of claim 1 wherein, the at least two monomeric catalyst components have tin-based centers that at least one bridging group is bonded to.

4. The catalyst composition of claim 2 wherein the at least one bridging group is an alkyl bridging group.

5. The catalyst composition of claim 2, of the type L₃Sn—X—SnL₃, where L can be taken from oxides, hydroxides, alkoxides, mercaptans, fatty acids, maleates, acetylacetonates, halides and mixtures therefrom; X is an alkyl bridge from C1 to C30, linear or branched.

6. A method of using the catalyst composition of claim 2 for esterification reactions.

7. A method of using the catalyst composition of claim 2 for urethane reactions.

8. A method of using the catalyst of claim 2 for esterification reactions.

9. A method of using the catalyst of claim 2 for urethane reactions.

10. A method of preparing a bridged catalyst composition, the method comprising the following steps:
charging a flask with NaOH and a surfactant;
fitting the flask with an addition funnel;
charging the addition funnel with Cl₃Sn(CH₂)₈SnCl₃ and an aromatic hydrocarbon;
heating the flask to a temperature and holding the flask at said temperature for a period of time forming a reaction slurry;
adding a rinsate to the reaction slurry, forming a top layer and a second slurry;
removing the top layer;
washing the second slurry;
filtering the second slurry;
rinsing the reaction flask was rinsed with a second rinsate resulting in a cake;
using the second rinsate to wash the cake resulting in a wetcake;
drying the wetcake to form a powder;
washing the powder with a third rinsate to form a slurry;
filtering the slurry to form a second wetcake;
drying the second wetcake at a second temperature for a second period of time, resulting in a second powder.

11. (canceled)

12. The catalyst composition of claim 1, of the type L₃Sn—X—SnL₃, where L can be taken from oxides, hydroxides, alkoxides, mercaptans, fatty acids, maleates, acetylacetonates, halides and mixtures therefrom; X is an alkyl bridge from C1 to C30, linear or branched.

13. The catalyst composition of claim 1, of the type L₃R—X—RL₃, where L can be taken from oxides, hydroxides, alkoxides, mercaptans, fatty acids, maleates, acetylacetonates, halides and mixtures therefrom; R is the nuclear center of the monomeric component; and X is an alkyl bridge from C1 to C30, linear or branched.

14. A method of using the catalyst composition of claim 1 for esterification reactions.

15. A method of using the catalyst composition of claim 1 for urethane reactions.
